(19) United States
(12) Patent Application Publication
(10) Pub. No.: US 2015/0216423 A1
(43) Pub. Date: Aug. 6, 2015

(54) METHOD FOR DIAGNOSING SELECTED CONDITIONS USING THERMOGRAPHY

(71) Applicant: Daniel Beilin, Aptos, CA (US)

(72) Inventor: Daniel Beilin, Aptos, CA (US)

(21) Appl. No.: 14/173,911

(22) Filed: Feb. 6, 2014

(51) Int. Cl. A61B 5/01; A61B 5/20; A61B 5/00

(52) U.S. Cl. CPC A61B 5/015; A61B 5/682; A61B 5/6822; A61B 5/6823; A61B 5/6824; A61B 5/4845; A61B 5/41; A61B 5/4227; A61B 5/425; A61B 5/4325; A61B 5/201

(57) ABSTRACT

Disclosed herein is a method of diagnosis for various conditions through use of a thermograph. Body temperatures measured at selected sites on the body observed before and after stimulus present data which when analyzed provides for reliable suspicions for selected conditions. Analysis of data includes several constructed indices and mathematical expressions used diagnose conditions of vulnerabilities to conditions.

### FIELD OF THE INVENTION

The present invention relates to the field of thermographic diagnosis. The present invention, more particularly, relates to the diagnosis of selected conditions which heretofore have not been accurately diagnosed using a thermograph.

### INCORPORATION BY REFERENCE

U.S. patent application Ser. No. 12/028,743 entitled, “Whole Body Infrared Thermography Systems and Methods” is incorporated by reference in its entirety and for all purposes to the same extent as if the patent application was specifically and reprinted here.

### BACKGROUND OF THE INVENTION

Thermography is used for measuring the amount of body heat delivered to the skin from a combination of a person's cellular metabolism and their nervous system in targeted areas of the body. These measurements may be taken from a number of sites on the body. Each site projects body heat for particular internal organs. The amount of heat projected represents the condition of that organ. When conducting thermographic diagnosis, an operator will take a first measurement of temperature data at selected sites then subject the patient to some stimulus. After the stimulus, temperature data is from the selected sites is taken a second time. Comparison of the first and second measurements at selected sites reveals the reaction of various organs to stimulus. That reaction is determinative to the diagnosis of various conditions.

Presently, many conditions which can be diagnosed by this method are well known and have been conducted by professionals regularly. These known conditions include heavy metal toxicity, cranial structural imbalance, hypothyroid conditions, sinus blockage, auxiliary lymph issues, food intolerance, and many more. Thermographic diagnosis is generally very non-invasive, cheap, and can be accomplished in a relatively short period of time. Therefore, using a thermograph to diagnose conditions is preferable when possible. However, this list of known diagnoses is limited. The thermograph is a dynamic device which has many undiscovered uses.

Accordingly, there is a need to establish methods for using a thermograph to diagnose a greater number of conditions. The present invention provides the necessary teachings to use a thermograph to diagnose conditions which previously needed to be diagnosed with other, less preferable methods.

### SUMMARY OF THE INVENTION

According to a first aspect of the method of the present invention a thermograph is used to collect temperature data from 120 sites on the body of a patient. The sites are grouped by region on the body. The patient is then asked to disrobe and allow their unclothed bodies adjust to the chill of room temperature. The chill causes a mini fight-or-flight reaction in the patient. A thermograph is once again used to take temperature data at the 120 sites. Each site has an expected normal variance in temperature over the course of the two readings. When sites do not show a normal change they are: blocked, hypo, hyper, or a paradox. Depending on the extent of that status, the status may be rated optimal, minimal, moderate, or severe. Based on the status rating at various sites and comparing that data to models, a technician can diagnose various conditions previously undiagnosable with a thermograph.

The thermography instrument used to measure temperature at the various sites on the body should be any device suitable to measure temperature of an isolated area of the body to a tenth of a degree. The temperature scale used is less important as long as it is consistent. The device used could be that as disclosed in U.S. patent application Ser. No. 12/028,743. Other thermography devices known in the art are equally acceptable provided that those devices had substantially similar or superior measurement capabilities.

### BRIEF DESCRIPTION OF THE DRAWING

For a further understanding of the objects and advantages of the present invention, reference should be had to the following detailed description, taken in conjunction with the accompanying drawing, in which like parts are given like reference numerals and wherein:

FIG. 1 is a report of data collected by a thermography device.

FIG. 2 is a close up of a human mouth demonstrating body sites relating to the teeth.

FIG. 3 is a portrayal of the human head demonstrating body sites relating to the head.

FIG. 4 is a close up of a human neck demonstrating body sites relating to the neck.

FIG. 5 is a portrayal of the human torso demonstrating body sites relating to the upper torso.

FIG. 6 is a portrayal of the human torso demonstrating body sites relating to the abdomen.

FIG. 7 is a close up of the human lower back demonstrating body sites relating to the lower back.

FIG. 8 is a portrayal of the human male breast demonstrating body sites relating to the breast.

FIG. 9 is a portrayal of the female breast demonstrating the manner in which breast body sites should be measured.

FIG. 10 is a portrayal of the female breast demonstrating how to find locate body sites on the breast.

FIG. 11 is a flow chart of the process used to diagnose selected conditions.

FIG. 12 is a flow chart illustrating the relation of a thermography device to the disorder index 25.

FIG. 13 is a flow chart illustrating the relation of a thermography device to the body chemistry vulnerability index.

### DETAILED DESCRIPTION OF THE INVENTION

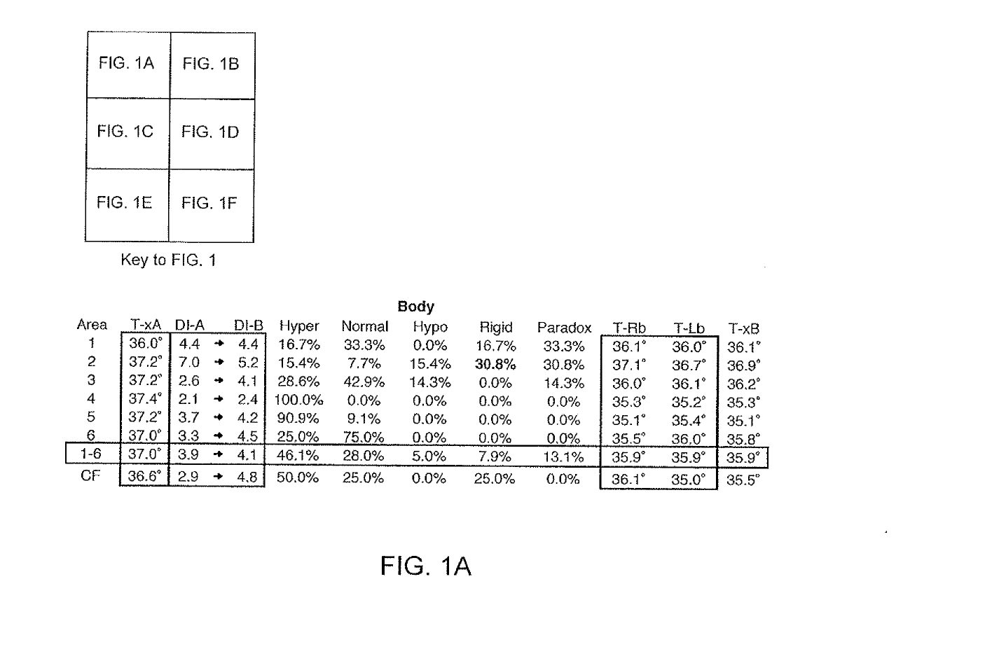

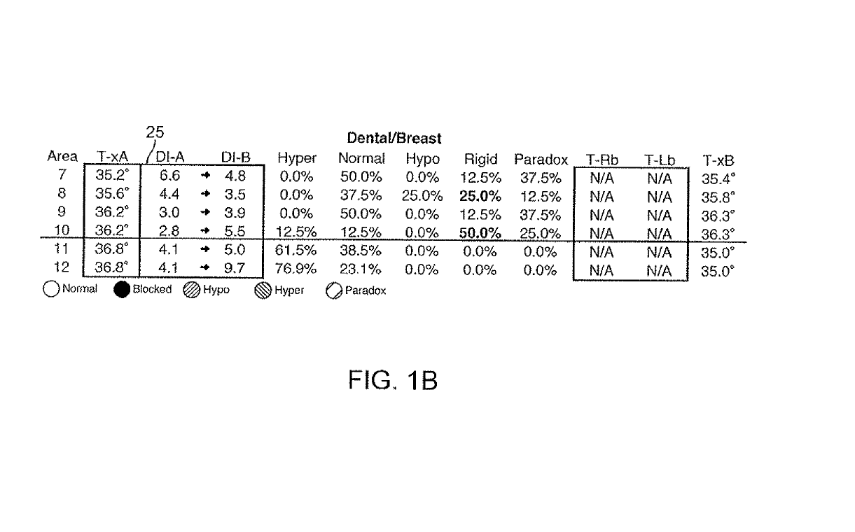

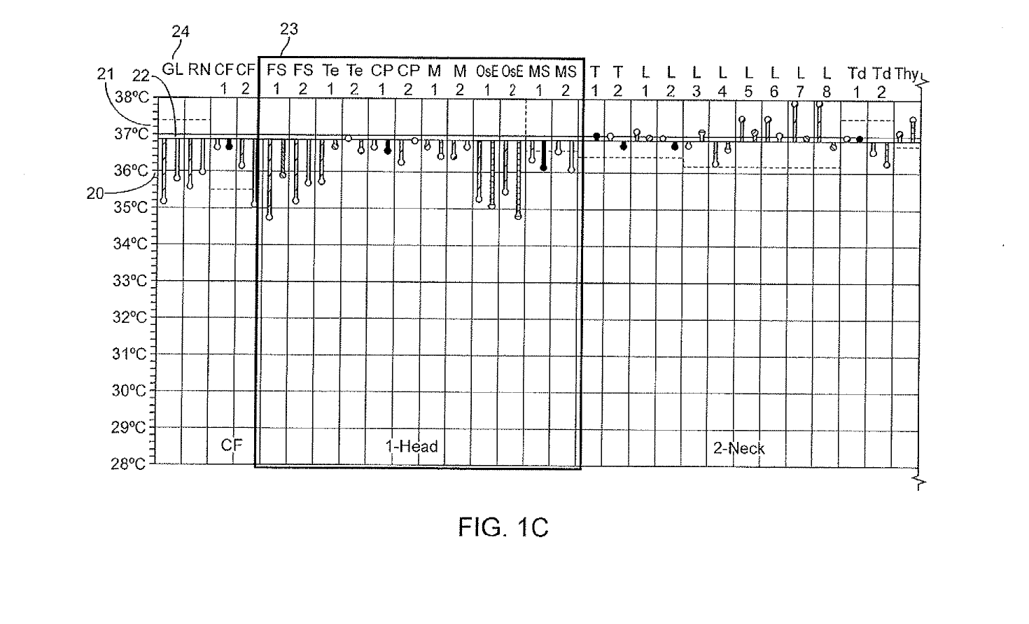

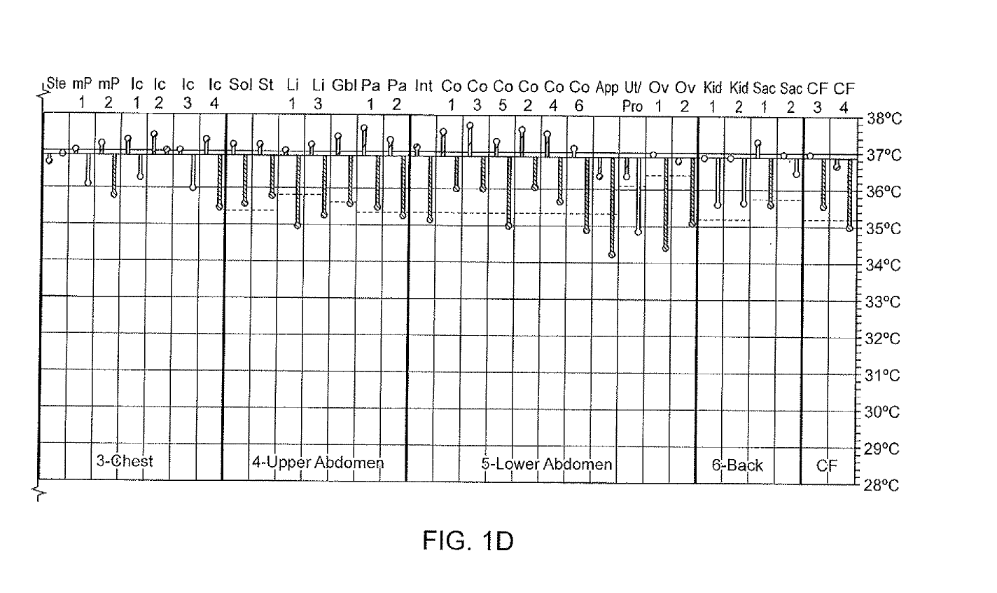

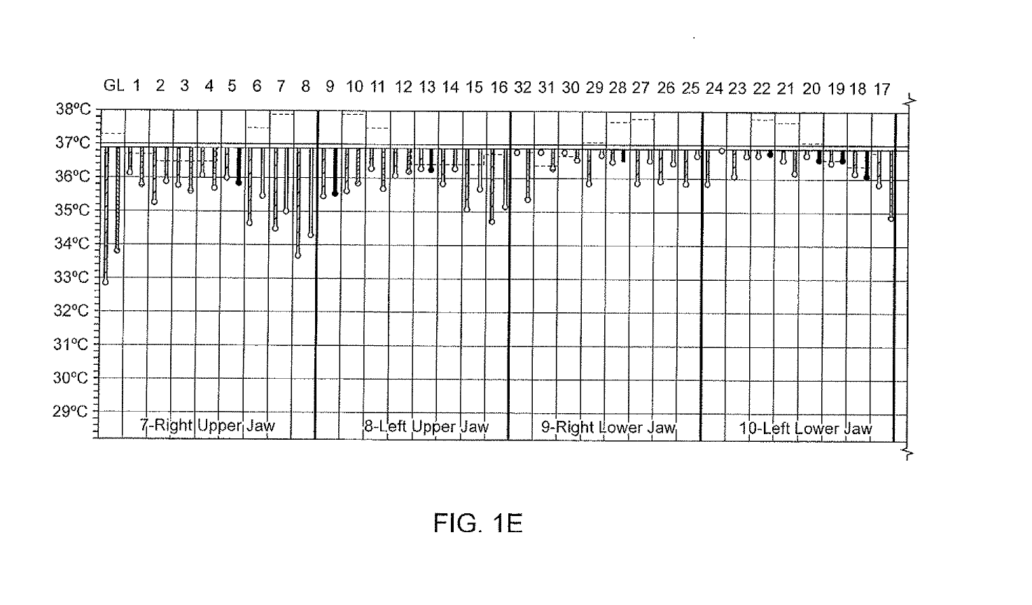

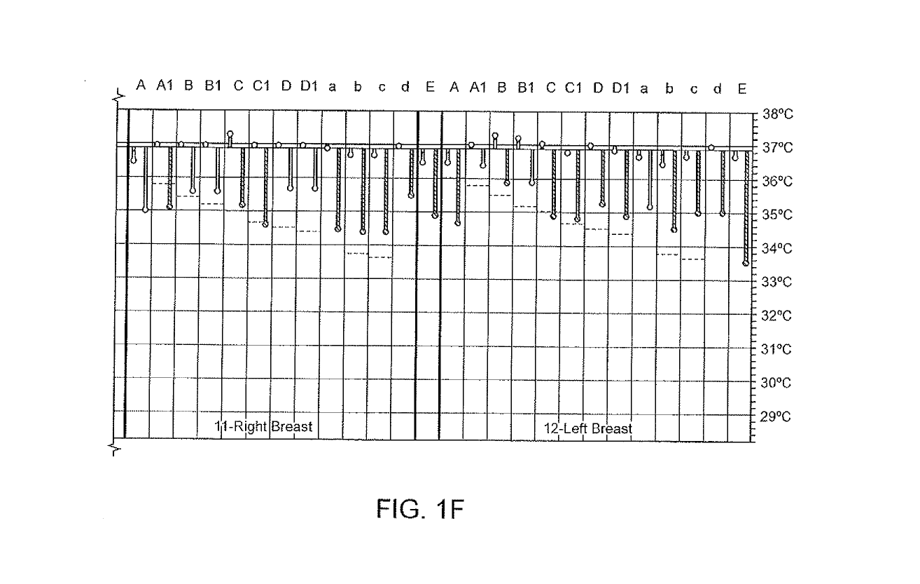

The invention will now be described in detail. First, with respect to FIG. 1, where FIG. 1 is a report of data collected by a thermography device, measurements are made twice. Measurements are performed before and after a brief cooling period (suggested 10 minutes) that acts as a mild stress stimulus, thereby inducing regulation through the autonomic nervous system's control of blood circulation to the skin. The report shows an initial reading 20 matched with stimulated reading 22 taken at a plurality of body sites 24. Though there are at least 120 body sites 24 which can be used in with the method of the present invention, the site identified by element number “24” on FIG. 1 refers to a location on the forehead of a patient. Nothing in this disclosure should be construed as stating that body sites 24 only refer to this site. Instead the element number refers to all of them. Each body site 24 will be detailed below. The body sites 24 are broken down in thirteen multi-site regions 23. The region identified by element number “23” refers to the head of the patient, but similarly to the body sites 24, the element number “23” refers to all of the thirteen multi-site regions.

By analyzing the skin's thermal responses to stress, insights can be gained into the physiological, functioning health of various systems of the body, in health and disease. After comparing the two measurements a given body site 24 is determined to be normal (the measure change is as expected), hypo (the measured change is less than expected), hyper (the measured change is greater than expected), a paradox (the measured change is opposite of expected), or blocked (little or no change measured). The conditions possible for each body site 24 will be discussed in greater detail below. An accurate test depends upon three criteria: Room Temperature, Test Schedule, and Patient Test Preparations.

Room temperature should be between 67° F. [19° C.] and 73° F. [23° C.]. If the room is too warm, the number of rigid/blocked sites will increase. Conversely, if too cold, the number of hyper-regulating sites measured will increase. Either case can reduce test accuracy by 40%-80%.

All tests should be completed before 2:00 P.M. The nervous system is sympathetically vital and responsive from morning to mid-afternoon. This is the optimum period to capture the responses from the autonomic nervous system through controlled, substantially standardized parameters. Accuracy may be reduced up to 30% when the test is performed in the late afternoon or evening.

The patient should follow all applicable test preparations. The patient should adhere to a given list of test preparations. Elements that aid accuracy include: The patient should wear comfortable, loose-fitting clothing that can be easily removed. The patient should avoid synthetic fibers and tight clothing. Female patients should not wear a bra or remove it at least 15 minutes prior to the test. The patient should not shower or bathe the morning of the test. However, a quick shower using only tepid water is allowable, as is a shower or bath the night before. The patient should refrain from using body sprays, skin creams or lotions, or cosmetics the morning of the test. The patient should not drink coffee, black tea, or caffeinated beverages, and not smoke the morning of the test. The patient should avoid hot food for breakfast, a light breakfast is acceptable. The patient should be hydrated; said patient should drink 12-16 oz of water ½-2 hours before the test. Patients should not exercise the morning of the test. This includes running, yoga, Pilates, etc. Patients should stop taking all non-essential supplements and remedies 24-36 hours prior to the test, but continue all prescription drugs. The patient should refrain from ‘regulative’ and therapeutic practices within 36 hours prior to the test. This includes acupuncture, bio-energetic treatments, therapeutic massage, classical homeopathy, chiropractic treatments, etc. The patient should refrain from dentistry and dental cleanings at least 3 days prior to the test. Female patients cannot be tested during the first or second day of the menstrual period. The lower abdominal sites warm up and create false readings. Finally the patient should not drink alcohol for at least 24 hours prior to the test.

Using the data collected, several other figures are constructed. These additional figures are used in diagnosis of selected conditions and vulnerabilities. The first is the baseline temperature 21. The baseline temperature 21 is the average of all the measurements taken in the initial reading 20 from all the body sites 24 except those pertaining to the breasts and the teeth. Though the format of the report in FIG. 1 is not a subject of this application, the body sites 24 relevant to the baseline temperature 21 are those found in the uppermost of the two rows in FIG. 1. The baseline temperature 21 is demonstrated on the report in FIG. 1 by the line where all other temperatures are displayed from. For illustrative purposes, in FIG. 1, the baseline temperature 21 is approximately thirty-seven degrees Celsius. This figure will of course vary between patients. The baseline temperature 21 will be referred to further below with respect to diagnosis.

Using the data grouped by the multi-site regions 23 an additional figure, known as the disorder index 25, is constructed known. The disorder index 25 is calculated per region, per data set; thus, there is a disorder index 25 reading for each of the thirteen multi-site regions 23 for both the initial reading 20 and the stimulated reading 22. The disorder index 25 is a rating system from one through ten. The disorder index 25 is calculated by taking the greatest difference in temperature readings for body sites 24 in a given region 23, and putting that delta into a mathematical formula specific to each region 23. If the mathematical formula provides a number greater than ten, ten is used. Table 1 below lists the mathematical formulas by multi-site region 23.

TABLE 1

| | |
|---|---|
| 1- Head | Disorder index = (9/8)(delta) + 1 |
| 2- Neck | Disorder index = (9/4)(delta) + 1 |
| 3- Chest | Disorder index = (9/5)(delta) + 1 |
| 4- Upper Abdomen | Disorder index = (9/5)(delta) + 1 |
| 5- Lower Abdomen | Disorder index = (9/5)(delta) + 1 |
| 6- Back region | Disorder index = (9/3)(delta) + 1 |
| 7- Right Upper Jaw | Disorder index = (9/4)(delta) + 1 |
| 8- Left Upper Jaw | Disorder index = (9/4)(delta) + 1 |
| 9- Right Lower Jaw | Disorder index = (9/4)(delta) + 1 |
| 10-Left Lower Jaw | Disorder index = (9/4)(delta) + 1 |
| 11-Right breast | Disorder index = (90/29)(delta) + 1 |
| 12-Left breast | Disorder index = (90/29)(delta) + 1 |
| 13-Cubital Fossa | If Delta > 1: Index = (20/9)(delta) + (50/9) |
| | If Delta = 1: Index = 7 |
| | If Delta < 1: Index = (25/4)(delta) + 1 |

A second index used in diagnosis is the body chemistry vulnerability index (BCV). This index is not displayed in FIG. 1. The result of the BCV is a single number ranging between one and ten. The greater the number of the BCV, the higher the body's risk of infection, or disorder development. The BCV is based upon data collected from six multi-site regions 23 of the body. The multi-site regions 23 includes are: the head, the neck, the chest, the upper abdomen, the lower abdomen, and the back. The body sites 24 that make up these multi-site regions 23 will be detailed below. To calculate the BCV, first find the greatest temperature difference between body sites 24 in each of the above listed regions from either measurement (either the initial measurement 20 or the stimulated measurement 24). This should result in 6 temperature deltas. Next, amongst those six temperature deltas, identify the greatest difference between deltas. For purposes of brevity, this figure which is the greatest difference between deltas will simply be referred to as “X.”

Based upon the value of X, one of five equations is used to act upon X in order to determine the BCV value. These equations are demonstrated in Table 2 below:

TABLE 2

| IF | THEN |
|---|---|
| 1 < X <= 3.6 | BCV = 1.25(X) − 0.5 |
| 3.6 < X < 4.2 | BCV = (5/3)(X) − 2 |
| 4.2 <= X <= 5.8 | BCV = 1.25(X) − 0.25 |
| 5.8 < X < 7 | BCV = (5/6)X + (13/6) |
| 7 <= X | BCV = X + 1 |

Once a test if performed, the results may suggest the diagnosis of certain selected conditions. Selected conditions as defined by this disclosure comprises: a toxicity index, a deficit in detoxification capability, immune system and body chemistry alert, adrenal stress, autoimmune disease, brain toxicity, thyroid hyperfunction or anomaly, hepatobiliary system stress, enzyme dysfunction/deficiency, Insulin resistance, Pancreas toxicity, pelvic toxicity, endometriosis in women, kidney hypo-function, mastopathy, breast asymmetry, tonsil block, sternum block, 2^{nd} molar block, stomach block, liver block, ovary dysregulation, dental toxicity, and dental foci suspicion. These conditions can be diagnosed in varying severity based upon data reading as seen below in Table 3. Following Table 3 is a description of how to interpret information contained in the table.

TABLE 3

| DISORDER AND | | | | |
|---|---|---|---|---|
| RECOMMENDATIONS | OPTIMAL | MINIMAL | MODERATE | SEVERE |
| Toxicity Index (colon, | | If 9 or more of | If 9 or more of the | If 9 or more of the |
| Kidneys, lungs hyper) | | the following | following sites are | following sites are |
| | | sites are hyper: | hyper: Int, Co 1-6, | hyper: Int, Co 1-6, |
| | | Int, Co 1-6, Ov1, | Ov1, Ov2, Kid1, Kid2, | Ov1, Ov2, Kid1, Kid2, |
| | | Ov2, Kid1, Kid2, | Sac1, Sac2. | Sac1, Sac2. |
| | | Sac1, Sac2. | AND | AND |
| | | | 4 or more of the | 4 or more of the |
| | | | following sites are | following sites are |
| | | | hyper: Sol, St, Li1, | hyper: Sol, St, Li1, |
| | | | Li3, Gbl, Pa1, Pa2 | Li3, Gbl, Pa1, Pa2 |
| | | | | AND |
| | | | | 4 or more of the |
| | | | | following points are |
| | | | | hyper: Ste, mP1, |
| | | | | mP2, Ic1, Ic2, Ic3, Ic4. |
| Detoxification Capacity | 0 or 1 | 2 systems | 3 systems blocked | 4 systems blocked |
| Lack (refers to kidneys, | systems | blocked | | |
| liver, gallbladder, | blocked | | | |
| lymph, and colon) | | | | |
| Note: a “system block” | | | | |
| means that at least one | | | | |
| site associated with a | | | | |
| particular organ is | | | | |
| blocked. | | | | |
| Immune System and | BCV and | If both BCV and | Either BCV or | Either BCV or |
| Body Chemistry Alert | immune | Immune index | immune index is | Immune index is |
| | index are | are greater than | greater than or | greater than or equal |
| | both less | or equal to 5, but | equal to 7 and less | to 9, and the other is |
| | than 5 or if | both less than 7. | than or equal to 9 | greater than or equal |
| | only one is | | and other is greater | to 8. |
| | less than or | | than or equal to 6 | |
| | equal to 5 | | | |
| Adrenal Stress Index | none | Difference | Difference between | Difference between |
| | | between initial | initial reading of | initial reading of |
| | | reading of | warmest tooth and | warmest tooth and |
| | | warmest tooth | coldest tooth (over | coldest tooth (over |
| | | and coldest tooth | all 32 teeth | all 32 teeth sites) >= 5 |
| | | (over all 32 teeth | sites) >= 4 | |
| | | sites) >= 3 | | |
| Autoimmune Indication | 100% hyper | 100% hyper in 2 | 100% hyper in 3 | 100% hyper in all 4 |
| (regions: Chest, Upper | in 1 or less | listed regions | listed regions | listed regions |
| and lower Abdomen, | listed | | | |
| and back) | regions | | | |
| Brain Toxicity | Half or less | 3 sites' stimulated | All 4 sites' | All 4 sites' stimulated |
| (Refers to sites FS1, | sites' | reading is at or | stimulated reading is | reading is greater |
| FS2, Te1, and Te2) | stimulated | below initial | at or below initial | than 0.3 below initial |
| | reading is at | reading +/− 0.1 | reading +/− 0.1 | reading. |
| | or below | | | |
| | initial | | | |
| | reading +/− 0.1 | | | |
| Thyroid | | Initial reading for | Initial reading for | Initial reading for |
| | | Td1 and Td2 less | Td1 and Td2 less | Td1 and Td2 less |
| | | than baseline | than baseline temp | than baseline temp |
| | | temp | AND | AND |
| | | AND | Stimulated reading | Stimulated reading |
| | | Stimulated | of either Td 1 or 2 is | of both Td 1 and 2 is |
| | | reading of either | less than or equal to | less than or equal to |
| | | Td 1 or 2 is less | baseline temp minus | baseline temp minus |
| | | than or equal to | 0.7 and remaining | 1 |
| | | baseline temp | site is less than or | |
| | | minus 0.4 and | equal to baseline | |
| | | remaining site is | temp minus 0.4 | |
| | | less than or equal | | |
| | | to baseline temp | | |
| | | minus 0.2 | | |
| Heptobiliary | | 1 of 3 of Li1, Li3, | 2 of 3 of Li1, Li3, Gbl | Li1, Li3, Gbl all |
| | | Gbl blocked or | blocked or paradox | blocked or paradox |
| | | paradox | | |
| | | OR | | |
| | | 1 of 3 of above | | |
| | | sites initial | | |
| | | reading is less | | |
| | | than baseline | | |
| | | temp plus 1.7 | | |
| Enzyme Dysfunction | | Pa1 initial reading | Pa1 initial reading is | Pa1 is blocked or |
| | | less than baseline | greater than | paradox AND Pa1 |
| | | temp minus 1.4 | baseline temp plus | initial reading is less |
| | | OR | 0.8 | than baseline temp |
| | | Pa1 initial value | AND | minus 1.4 |
| | | greater than | Pa1 is hypo, blocked, | OR |
| | | baseline temp | or paradox | Pa1 initial reading is |
| | | plus 0.4 AND Pa1 | | greater than baseline |
| | | is blocked or | | temp plus 1.5 AND |
| | | paradox | | Pa1 is hypo, blocked |
| | | | | or paradox |
| Insulin Resistance | | Pa2 initial reading | Pa2 initial reading is | Pa2 initial reading is |
| | | is 1.5-1.9 below | less than or equal to | less than or equal to |
| | | baseline temp | baseline temp minus | baseline temp minus |
| | | | 2 | 2 |
| | | | OR | OR |
| | | | Pa2 is blocked (0.1 degree | Pa2 is blocked (0.1 degree |
| | | | variation is | variation is |
| | | | acceptable). | acceptable) AND Pa2 |
| | | | | initial reading is less |
| | | | | than baseline temp |
| | | | | minus 2 |
| Pancreas Toxicity | | Pa1 or Pa2 where | Pa1 or Pa2 where | Pa1 or Pa2 where the |
| | | the difference | the difference | difference between |
| | | between initial | between initial | initial reading and |
| | | reading and | reading and | stimulated reading is |
| | | stimulated | stimulated reading is | greater than 2.2 |
| | | reading is greater | greater than 1.8 | |
| | | than 1.3 | | |
| Pelvic Toxicity | | Amongst Ut/pro, | Amongst Ut/pro, | Amongst Ut/pro, |
| (Endometriosis) | | Ov1, and Ov2, | Ov1, and Ov2, two | Ov1, and Ov2, all |
| | | two of the three | of the three cool | three cool more than |
| | | cool more than | more than 2.9 degrees | 2.9 degrees from |
| | | 2 degrees from | from initial | initial to stimulated |
| | | initial to | to stimulated | reading |
| | | stimulated | reading | |
| | | reading | | |
| Kidney Hypo-function | | Kd1 OR Kd2 | Kd 1 OR Kd2 initial | Kd1 AND Kd2 initial |
| | | initial reading is | reading is below | reading is below |
| | | below baseline | baseline temp by | baseline temp by |
| | | temp by 1.4-1.7 | more than 1.7 | more than 1.5 |
| Organ-Tissue Influence | | | | |
| Distant/Related Focal | | | | |

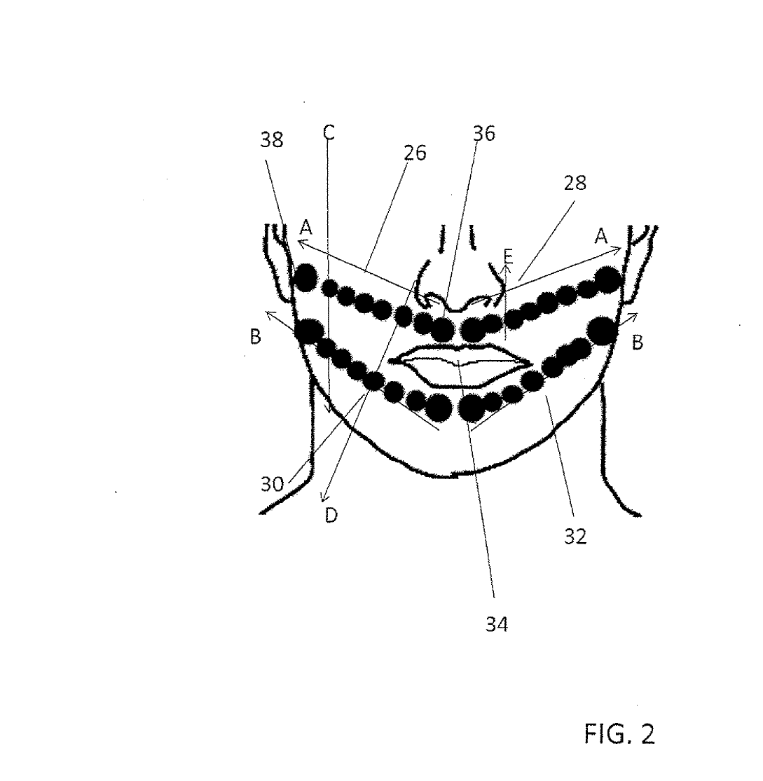

Explanations on how to find the body sites 24 and how to read the shorthand in Table 3 will now follow. With respect to FIG. 2, there is shown a close up of a human mouth demonstrating body sites relating to the teeth. The teeth are divided into four dental areas, or quadrants. Each quadrant is measured one at a time with 8 measurements per quadrant. The quadrants are measured in the following order: (1) upper-right quadrant 26, (2) upper-left quadrant 28, (3) lower-right quadrant 30, and (4) lower-left quadrant 32. Each of these quadrants represents a region for the disorder index 25. The teeth are measured with the mouth closed 34. Measurements of each quadrant begin on the skin and at the gumline over the central incisor (front tooth) 36 progressing back towards the skin over the back molars 38. If a thermograph device which is not site specific is used to take measurements, a single measurement which includes all these sites is sufficient. To understand Table 3 and the data sheet as pictured in FIG. 1, each tooth body site 24 has been given a number designation, 1-32. The number in each quadrant where the upper-right quadrant 26 includes teeth 1-8 in descending order, the upper-left quadrant 28 includes teeth 9-16 in ascending order, the lower-right quadrant 30 includes teeth 25-32 in ascending order, and the lower-left quadrant 32 includes teeth 17-24 in descending order.

All teeth sites must be measured, even if one or more teeth have been extracted or there are root canals, bridges, or implants. Reflections of the retromolar space as well as latent infections due to cavitations can be crucial in particular case analyses. When measuring the upper quadrants, each site resides along the line from the middle of the philtrum to the tragus (Line A), with the final site falling along the anterior edge of the masseter muscle (Line C). It is helpful to make a small mark with a felt pen in front of the masseter muscle and plan that mark to be the last site of measurement for the quadrant. The line of measurement of the teeth is a straight line and each site has a circumference that is sequentially contiguous with the next site in the quadrant. For the lower quadrants, each site resides along the line from the central incisor, just above the jaw bone, to the earlobe (Line B). The first four dental sites are about ¼-⅜ in apart [⅝-1 cm], and are usually located before the mouth crease, with the fourth tooth (first premolar) on the nasolabial fold (Line D). The last four teeth are about ⅜-½ in apart [1-1¼ cm], with the last tooth just in front of the masseter muscle (Line C). Also, for the upper quadrants 26 and 28, the third tooth (canine) is usually located along the vertical edge of the nasal wing (Line E). Dental sites are measured on the skin over the area where the tooth enters the gums.

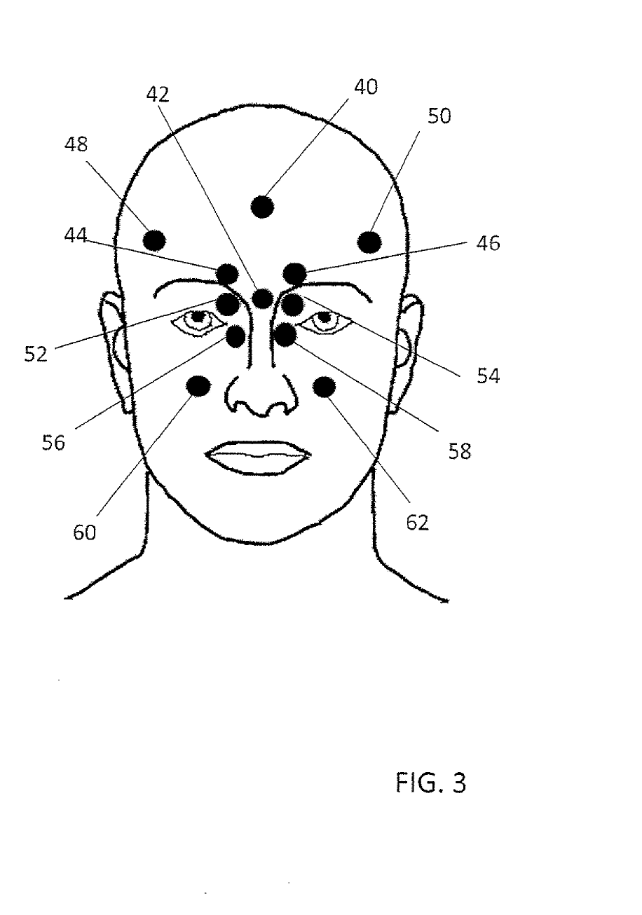

With respect to FIG. 3, there is shown portrayal of the human head demonstrating body sites relating to the head and the head region as referred to in Table 1. There are twelve body sites 24 on the head in addition to the thirty-two teeth sites. The first site is the glabella, abbreviated “GL” 40. This measuring spot is on the forehead, in the center, about ⅓ the distance from the frontal eminence to the top of the forehead, in a small indentation. The glabella is measured 3 times in a row at the beginning of the test, and only one time at the start of the second set of measurements. The next site is the Radix Nasi, abbreviated “RN” 42. RN 42 is found in the center of bridge of the nose, in the lowest part of the bridge. The next site(s) are parts of the frontal sinus, and are symmetrical sites which are abbreviated “FS1” 44 and “FS2” 46. The frontal sinus sites are located just above the medial (innermost) edge of the eyebrow. The next set of sites are the temples, and are symmetrical sites which are abbreviated “TE1” 48 and “TE2” 50. Dividing the temple into thirds front-to-back, these sites lay on the front third dividing line and in the middle of the temple vertically. The next set of sites are the commissura palpabrum, and are symmetrical sites which are abbreviated “CP1” 52 and “CP2” 54. The commissura palpabrum sites are found in the corner soft-tissue depression above the inner canthus of the eye, below the medial edge of the eyebrow, and angled up and medially toward the Glabella. The next set of sites are the ethmoid sinus, and are symmetrical sites which are abbreviated “OsE1” 56 and “OsE2” 58. On the side of the bridge of the nose, at the small depression just above where eyeglasses usually sit. The last set of sites on the head are the maxillary sinuses, and are symmetrical sites which are abbreviated “MS1” 60 and “MS2” 62. The maxillary sinuses are found at the intersection of a vertical line directly down from the pupil of the eye and the horizontal line of the side of the nostril, on bottom edge of the cheekbone.

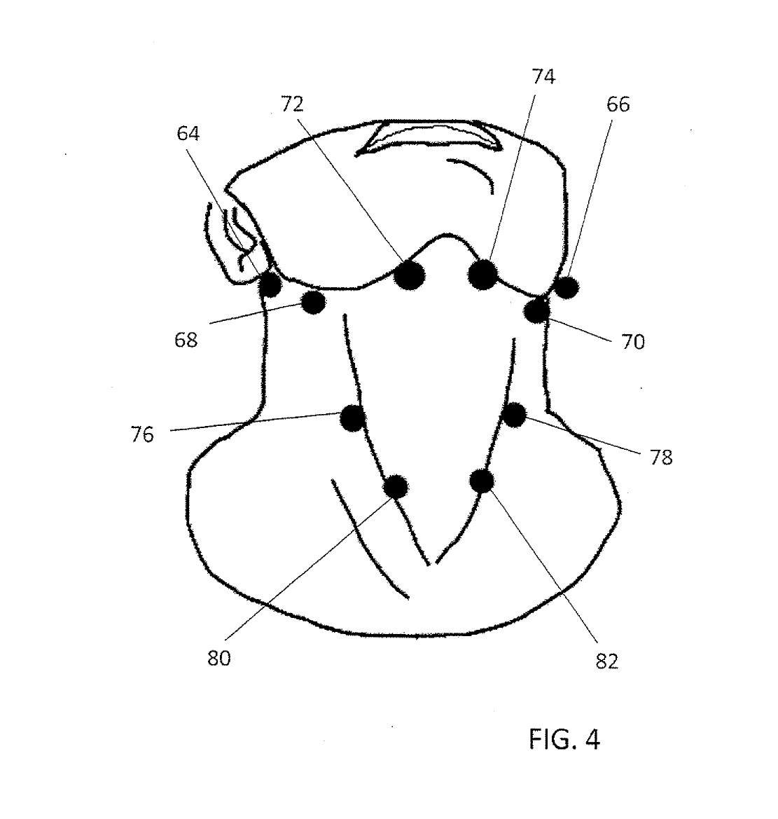

With respect to FIG. 4, there is shown a close up of a human neck demonstrating body sites relating to the neck. This figure shows the majority of the sites relating to the neck region as referred to in Table 1. The first set of sites are the mastoids, and are symmetrical sites which are abbreviated “M1” 64 and “M2” 66. Behind the earlobe and directly on top of the mastoid bone. The next set of sites are the tonsils, and are symmetrical sites which are abbreviated “T1” 68 and “T2” 70. The tonsils are found under the end of the lower jaw, in the soft tissue. This is best found at the apex of the angle of the lower jaw, underneath, in the soft tissue. The next set of sites are the inframandibular lymph nodes, and are symmetrical sites which are abbreviated “L1” 72 and “L2” 74. The inframandibular lymph nodes are found in the soft tissue below the central lower jaw, midway between the angle of the jaw and the chin. The next set of sites are the ventro sternocleidomastoids, and are symmetrical sites which are abbreviated “L3” 76 and “L4” 78. The ventro sternocleidomastoids are found at the front of the sternocleidomastoid muscle, in the center of its full length. The final set of sites in the neck region is the thyroids, and are symmetrical sites which are abbreviated “Td1” 80 and “Td2” 82 The thyroid is found ⅜ in [1 cm] lateral the midline, on the thyroid gland ¾ in [2 cm] above the depression at the suprasternal notch, about ¼-⅓ distance of neck, to the side of the center line in front of the sternocleidomastoid muscle.

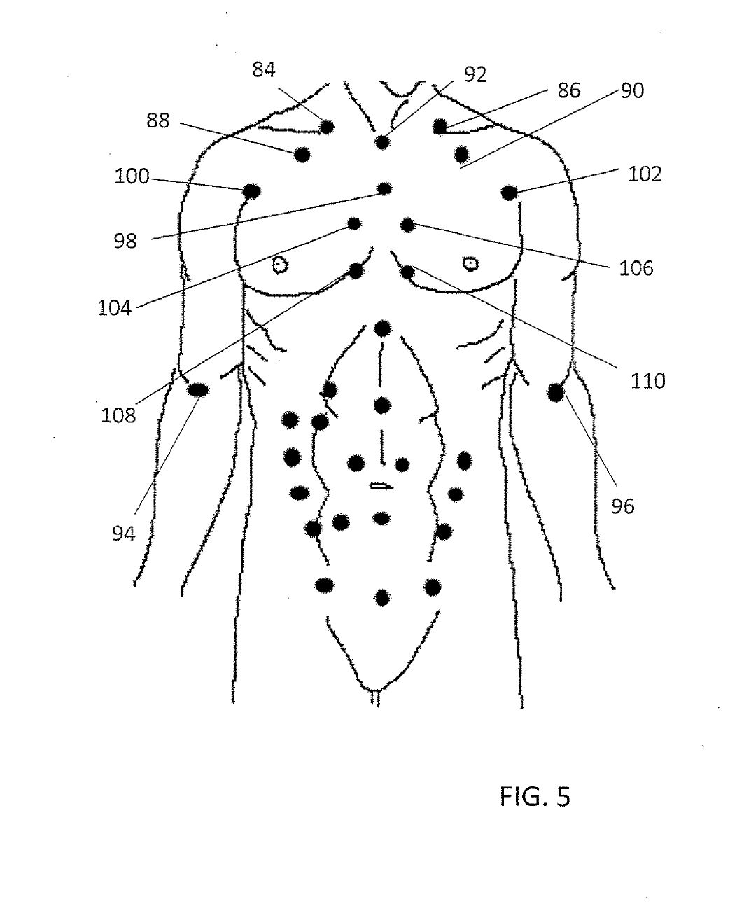

Referring now to FIG. 5, there is shown a depiction of the human torso demonstrating body sites relating to the torso. The first set of sites are the supraclavicular fossa, and are symmetrical sites which are abbreviated “L5” 84 and “L6” 86. The supraclavicular fossa are found In the middle of the depression above the medial end of clavicle. The patient can move shoulders slightly forward to see this depression clearly. The next set of sites are the infraclavicular fossa, and are symmetrical sites which are abbreviated “L7” 88 and “L8” 90. The infraclavicular fossa are found below the lateral end of the clavicle, in the depression. The patient can move shoulders slightly forward to see this depression clearly. The next site is the thymus, and it is abbreviated “Thy” 92. The thymus is found directly in the fossa above the sternum (above the suprasternal notch), forming a downward-facing triangle with thyroid sites. These above sites, though found on FIG. 5 are also included in neck region as referred to in Table 1.

The next set of sites show on FIG. 5 are the cubital fossa, and are symmetrical sites which are abbreviated “CF1,3” 94 and “CF2,4” 96. The cubital fossa are found at the inside crease of the elbow. The sites 94 and 96 should not be measured on top of a vein, always to either side but it is good to mark this site as it is crucial to repeat at the same location later in the testing procedure (hence two numbers in each abbreviation). The Cubital Fossa are their own region for purposes of Table 1.

The next site shown on FIG. 5 is the sternum, and is abbreviated “Ste” 98. The sternum site is found on the breastbone, at the boney protrusion exactly on the center line, ⅓ the distance downward from the suprasternal notch to the xyphoid process. The next set of sites are the lateral pectoralis major muscles, and are symmetrical sites which are abbreviated “mP1” 100 and “mP2” 102. The lateral pectoralis major site is found above the frontal end of axillary fold, ½-1 in [1½-2½ cm] above the crease on each side. The next set of sites are known as the intercostal space III, and are symmetrical sites which are abbreviated “Ic1” 104 and “Ic2” 106. The intercostal space III is found at the third intercostal space, between the 3rd and 4th ribs, about 1¼ in [3 cm] from the center line. The next set of sites are known as the intercostal space V, and are symmetrical sites which are abbreviated “Ic3” 108 and “Ic4” 110. The intercostal space V is found at the fifth intercostal space, between the 5th and 6th rib, 1¼ in [3 cm] from the center line. The sites discussed in this paragraph are affiliated with the chest region as referred to in Table 1.

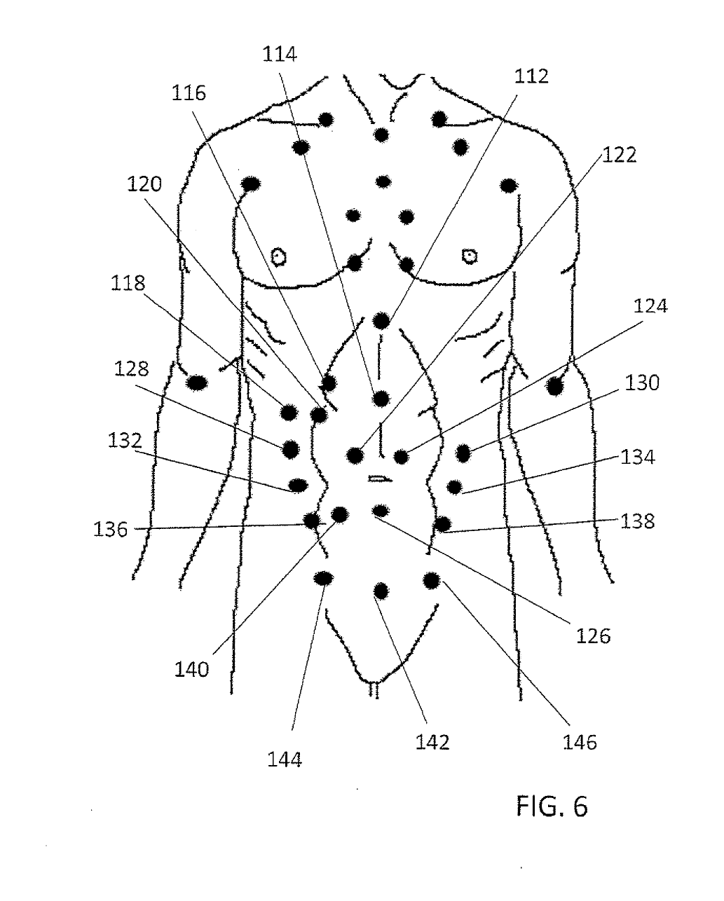

With particular reference to FIG. 6, it is understood that in general it is the same as FIG. 6 with the exception that this illustration it is directed to the lower body sites 24 affiliated with the upper and lower abdomen regions as referred to in Table 1. First, this disclosure will go over the sites included in the upper abdomen. The first upper abdomen site is the solar plexus, and it is abbreviated “So1” 112. The solar plexus site is found centered below the tip of the xyphoid process (cartilage extending from the lower meeting site of the ribs and sternum). The next site on the upper abdomen is the stomach, and this site is abbreviated “St” 114. The stomach site is found half way between the xyphoid process and navel, on the center line. The next site is the first liver site, and is abbreviated “Li1” 116. The liver site is found at the right sub-costal border, 2 in [5 cm] lateral from the center line. The next site is the second liver site and is abbreviated “Li3” 118. The second liver site is found at the right sub-costal border, 3½ in [9 cm] lateral from Li1 116. The next site is the gall bladder, and is abbreviated “Gb1” 120. The gall bladder site is found between both liver sites and downward 2 in [5 cm] below the middle of the liver sites, forming an equilateral triangle. The next set of site are the pancreas head and tail, and are symmetrical sites which are abbreviated “Pa1” 122 and “Pa2” 124. The Pancreas sites are located two-thirds the way from the stomach site and navel, 1½ in [3½ cm] lateral the center line, on either side.

Still referring to FIG. 6, the following sites are affiliated with the lower abdomen region as referred to in Table 1. The first lower abdomen site is the intestine and is abbreviated “Int” 126. The intestine site is located 1½ in [3½ cm] below the navel on the center line. There are six sites for the colon. The first pair of colon sites are abbreviated “Co1” 128 and “Co2” 130. The first two colon sites are symmetrical and are found directly below the nipple, 1½ in [3½ cm] under the costal border. The second pair of colon sites are abbreviate “Co3” 132 and “Co4” 134. The second pair of colon sites are symmetrical and are found 2-3 in [5-7½ cm] directly below the Co1/2 128/130 site and nipple, level with the navel. The last pair of colon sites are abbreviated “Co5” 136 and “Co6” 138. The last pair of colon sites are symmetrical and are found Directly below the Co1/2 128/130 and Co3/4 132/134 sites, about 2 in (5 cm) below Co3/4, level with the anterior iliac crest.

The remaining lower abdomen sites are found on FIG. 6. The next site is for the appendix and is abbreviated “App” 140. The appendix site is located in the middle of the area between prominence of the hip bone (anterior iliac crest) and the navel. The next site is based on gender but is located in the same location regardless. This site is either for the uterus or the prostate and is abbreviated “Ut/Pro” 142. The uterus or prostate site is located on the center line, directly above pubic bone and hairline. The location of the last two sites differ between males and females, though the abbreviations remain the same. In females, the last set of sites refer to the ovaries, and are symmetrical sites abbreviated by “Ov1” 144 and “Ov2” 146. The ovary sites are located 1 in [2½ cm] above the uterus/prostate site and 2 in [5 cm] out from the center line. In men, the sites with the same abbreviations are for the inguinal lymph, and these sites are found at the inguinal crease at the groin area at the same level as the prostate site.

With particular reference to FIG. 7, there is illustrated a close up of the human lower back demonstrating body sites relating to the lower back and are affiliated with the back region as referred to in Table 1. The lower back region has 2 pairs of symmetrical sites. The first set is for the kidney, and the symmetrical sites are abbreviated “Kid1” 148 and “Kid2” 150. The kidney sites are found below the last rib, about 2 in [5 cm] from the center of the spine. The second pair of sites are at the sacroiliac joint and are abbreviated “Sac1.” 152 and “Sac2” 154. The sacroiliac joint sites are found on the lower back, in the small indentation (dimple) on the superior aspect of the sacrum, about 2 in [5 cm] out from the midline.

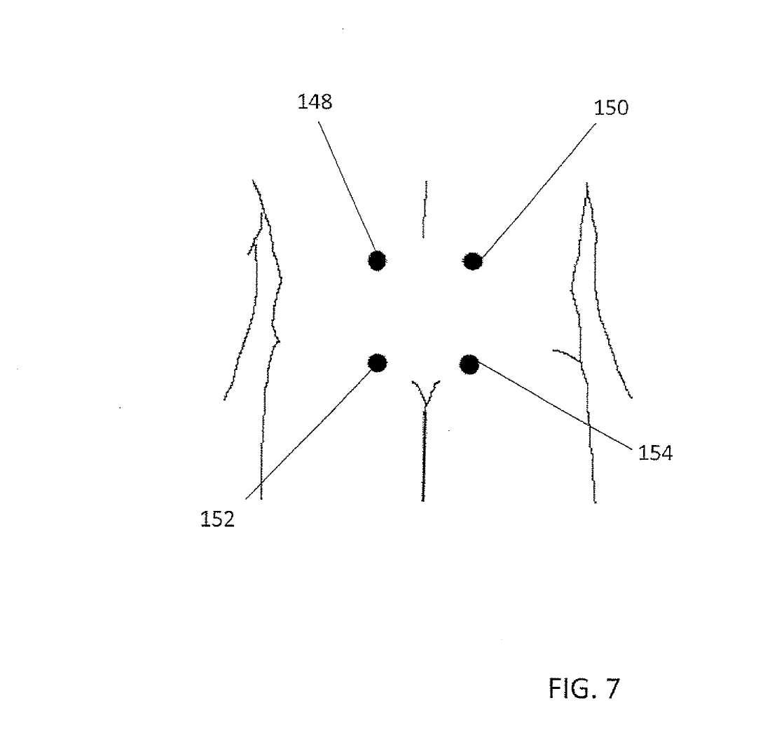

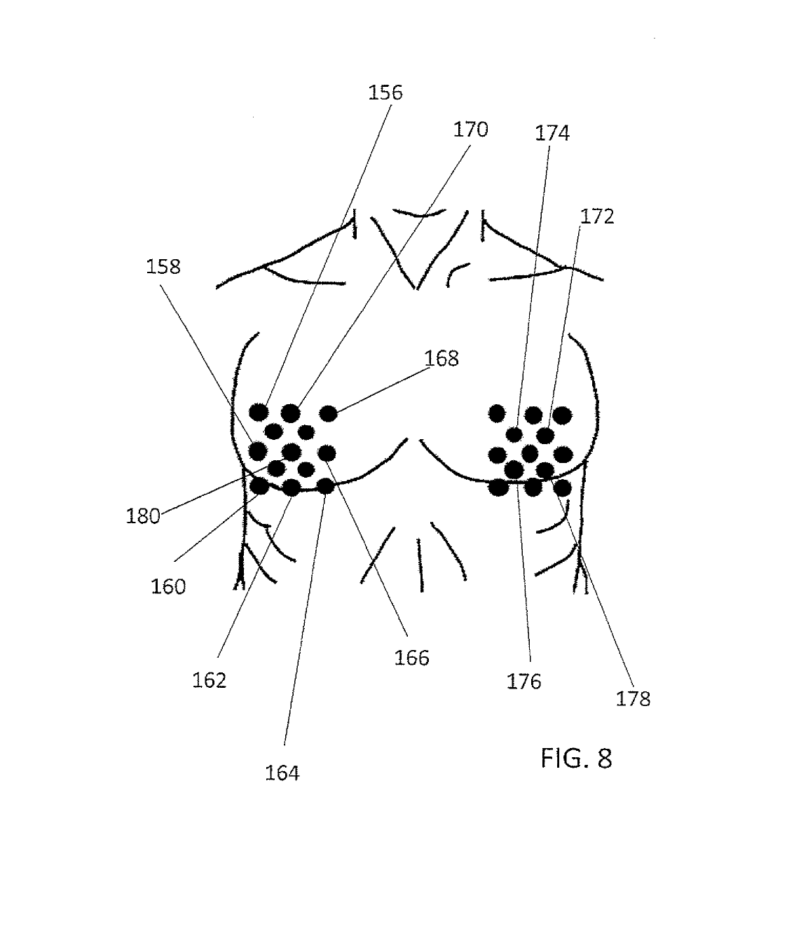

With particular reference to FIG. 8, there is illustrated a portrayal of the human male breast demonstrating body sites relating to the breast. There are 13 body sites 24 on each breast. The 13 sites are abbreviated beginning with the highest outer site as “A” 156, then following a circular pattern on the outermost sites proceeding down from “A” 156 the sites are “A1” 158, “B” 160, “B1” 162, “C” 164, “C1” 166, “D” 168, and “D1” 170. The inner grouping of body sites 24 follows the same pattern, beginning at the highest outermost site, the sites are abbreviated “a” 172, “b” 174, “c” 176, and “d” 178. The site at the nipple is abbreviated “E” 180. Because the 13 breast sites are identified by beginning on the outside and circling down and in, the sites on each breast, right and left, are mirror images of the other.

Breasts come in various shapes and sizes. However, the following pattern will ensure consistency, not only between the first and second set of measurements, but also from one test to the next. Large, pendulous breasts may affect the lower breast sites, B 160, B1 162, and C 164, as well as the liver sites (Li1 116, Li3 118). In such cases, measurements should only be done on skin exposed to air and as close as possible to the actual site location; a large breast should not be lifted to test the skin underneath. If the patient has had a mastectomy or has breast implants, breast measurements should still be taken, while avoiding the scars. Also because of the various shapes of breasts, areola size, and location, some sites may be in close proximity, such as B 160, B1 162, and b 174. It is important to measure as close to the site locations as possible, while being consistent between the first and second sets of measurements.

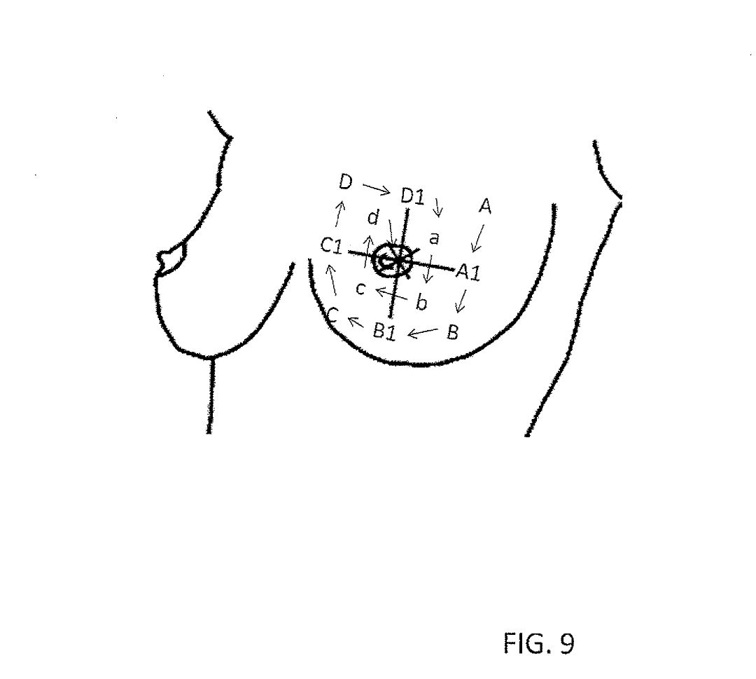

With particular reference to FIG. 9, there is illustrated a portrayal of the female breast demonstrating the manner in which breast body sites should be measured. Assuming use of a thermograph that measures single sites at a time rather than whole regions, the data from each breast (having the same sites 156-180 on both males and females) should be taken in a circular fashion starting from the first, outer, uppermost site 156, then proceeding down and in a circular motion towards the nipple 180. Each site can be located easily by imagining 2 crosses, one vertical, and one diagonal centered on the nipple.

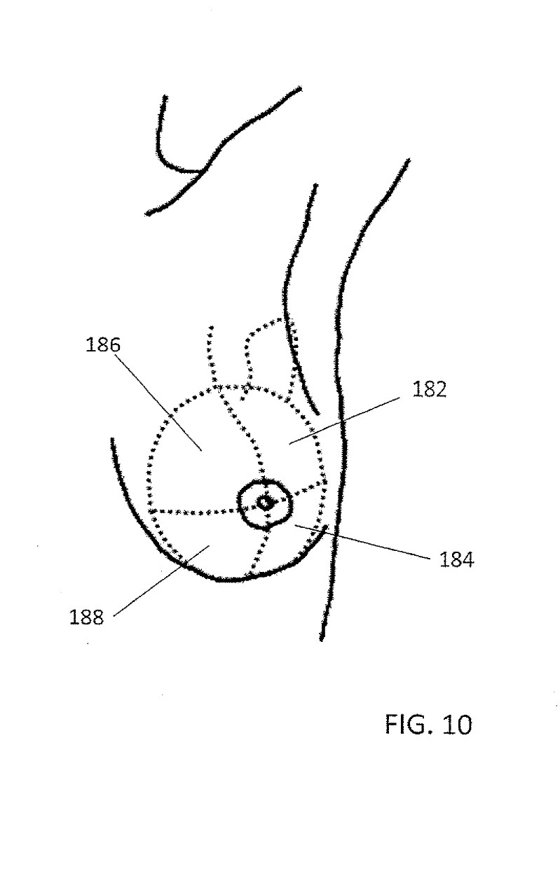

With particular reference to FIG. 10, there is illustrated the locations of each of the body sites 24 in the breast region of FIGS. 8 and 9. Each breast is divided into four quadrants: Upper-Outer 182 and Lower-Outer 184 (lateral) and Upper-Inner 186 and Lower-Inner 188 (medial). Site A 156 is found at the median of, and inside, the rim of the Upper-Outer quadrant 182. Site A1 158 is found on the boarder of the Upper-Outer 182 and Lower-Outer quadrants 184, in line with sites A 156 and B 160. Site B 160 is found at the median of, and inside, the rim of the Lower-Outer quadrant 184. Site B1 162 is found on the boarder of the Lower-Outer 184 and Lower-Inner quadrants 188, in line with sites B 160 and C 164. Site C 164 is found at the median of, and inside, the rim of the Lower-Inner quadrant 188. Site C1 166 is found on the boarder of the Upper-Inner 186 and Lower-Inner quadrants 188, in line with sites C 164 and D 168. Site D 168 is found at the median of, and inside, the rim of the Upper-Inner quadrant 186. Site D1 170 is found on the boarder of the Upper-Inner 186 and Upper-Outer quadrant 182, in line with sites D 168 and A 156.

Site a 172 is found in the Upper-Outer quadrant 182, 1/4 in [1 cm] outside the areola. Site b 174 is found in the Lower-Outer quadrant 184, 1/4 in [1 cm] outside the areola. Site c 176 is found in the Lower-Inner quadrant 188, 1/4 in [1 cm] outside the areola. Site d 178 is found in the Upper-Inner quadrant 186, 1/4 in [1 cm] outside the areola. Finally, site E 180 is on the nipple, or at its base if the nipple is long.

Another figure referred to in Table 3 is the immune index. The immune index is constructed by the data collected and is a single number ranging from zero to nine. A greater number indicates an immune system vulnerability. To calculate, initially start with an immune index figure of five. Then, find the number of blocked sites within sites T1-2 68,70 and L1-8 72-78, 84-90. For this purpose “blocked” means a change of 0.2 degrees or less. If there are no blocked sites amongst the above group, subtract three from the immune index figure. If there are one to three blocked sites, add one to the immune index figure. If there are four or more blocked sites, add two to the immune index figure. Next, if the thymus (Thy) cools 0.2 degrees or more, subtract two from the immune index figure. For purposes of this index, Thy is blocked if the site falls within the range of cooling by 0.1 degrees or warming by 0.2 degrees. For purposes of this immune index, Thy is paradox if it warmed by more than 0.2 degrees. If Thy is blocked and the initial measurement 22 for Thy is greater than or equal to the baseline temperature 21 plus 0.4 degrees, add two to the immune index figure. If Thy is blocked and the initial measurement 22 for Thy is less than the baseline temperature 21 plus 0.4 degrees, add one to the immune index figure. If Thy is paradox, and the initial measurement 20 for Thy is greater than the baseline temperature 21, add one and a half to the immune index figure. If Thy is paradox and the initial measurement 20 for Thy is equal to or less than the baseline temperature 21, add one to the immune index figure. After running through all those conditions the resulting immune index figure will be a number between zero and nine.

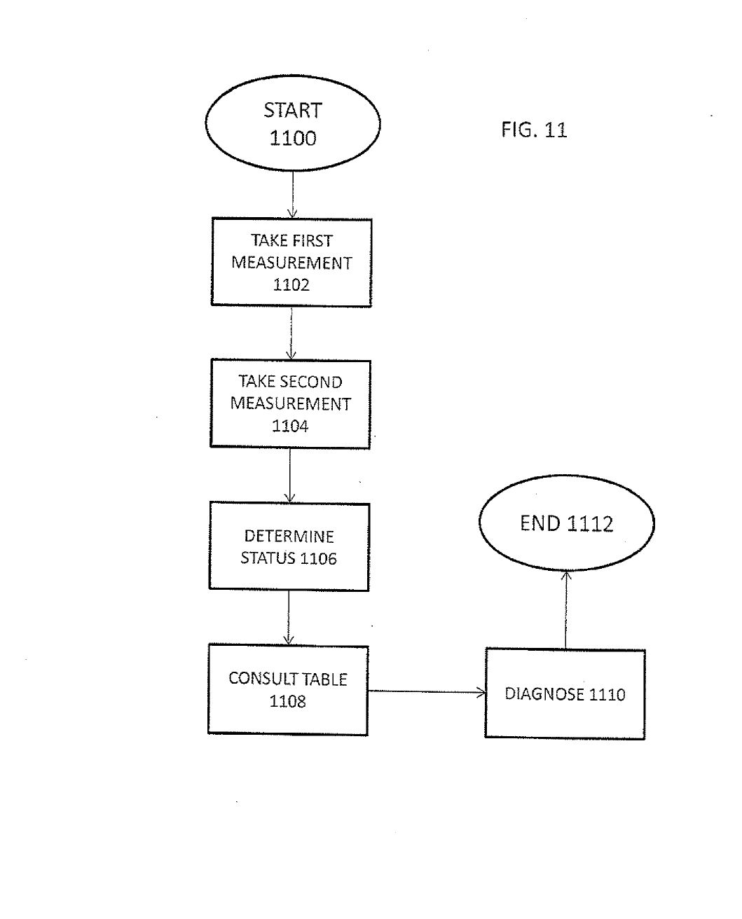

With particular reference to FIG. 11, there is illustrated a flow chart of the process used to diagnose selected conditions. First a technician will use a thermography device to take an initial measurement 20 of the body temperature at all of the sites described above (1102). Then stimulus is applied to the patient, and the thermography device is used to take a second stimulated measurement 24 (1104). Each site is then assigned the label normal, blocked, hypo, hyper, or paradox (1106). It is suggested that normal be defined as a site having the expected change and blocked as having no change, each having a range of a tenth of degree of variance either positive or negative. In certain circumstances, two tenths a degree of variance either negative or positive are acceptable. The remaining labels are defined by temperature ranges with respect to blocked and normal wherein: hyper falls above normal, hypo falls between blocked and normal, and paradox falls beyond blocked in the reverse direction (cooling or warming) of normal. Once the data is collected and the sites are labeled, consult Table 3 for matching data (1108). Once appropriate table entries are located, assess severity and treat accordingly (1110).

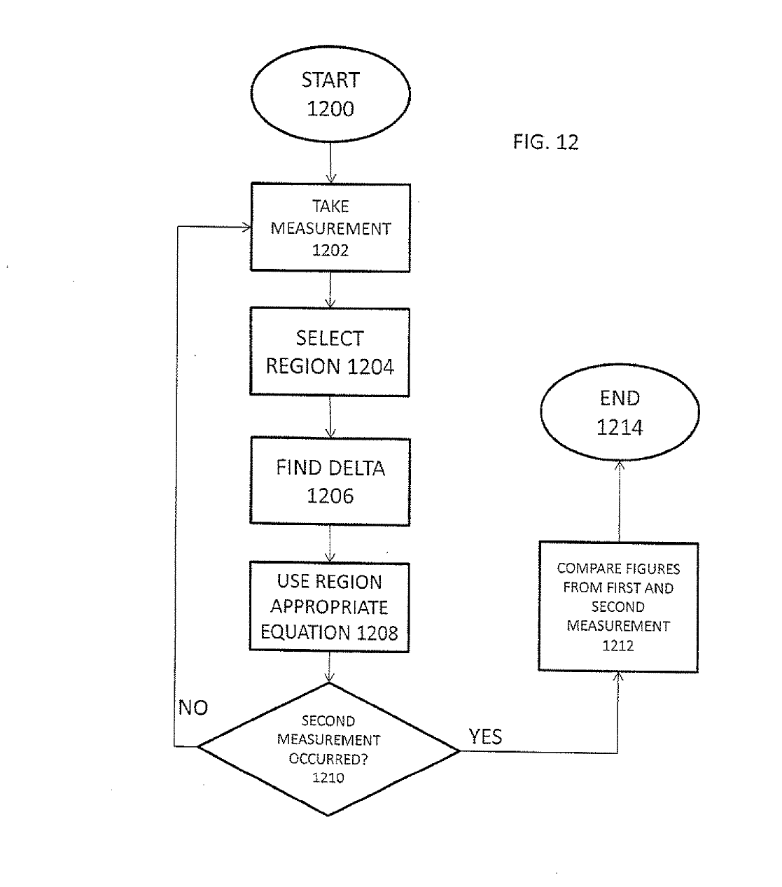

With particular reference to FIG. 12, there is illustrated a flow chart illustrating the relation of a thermography device to the disorder index 25. The disorder index, will have a figure for each region and each measurement. Thus, first the initial measurement 20 needs to be taken (1202). Then, one must focus on a particular multi-site region 23, such as the head (1204). Next, one must find the greatest delta between temperatures of that multi-site region 23 (1206). Given the delta, the delta is substituted into the correct equation displayed into Table 1 providing a result (1208). Then the same process is repeated for the stimulated measurement 24 (1210). Once a technician or user has all the necessary figures for the disorder index 25, these figures are compared (1212). Comparing the disorder index 25 figure from a particular multi-site region 23 from the initial reading 20 to the same figure from the stimulated reading 24 informs a technician the level of “order” within a particular multi-site region 23. The disorder index 25 thereby allows one to assess the preparedness of body systems or regions to respond to stimuli. If the disorder index 25 increases from the initial reading 20 to the stimulated reading 24, a technician will know that the system or region is problematic. Depending on the severity of the discrepancy in disorder figures, additional tests targeting the region should be ordered.

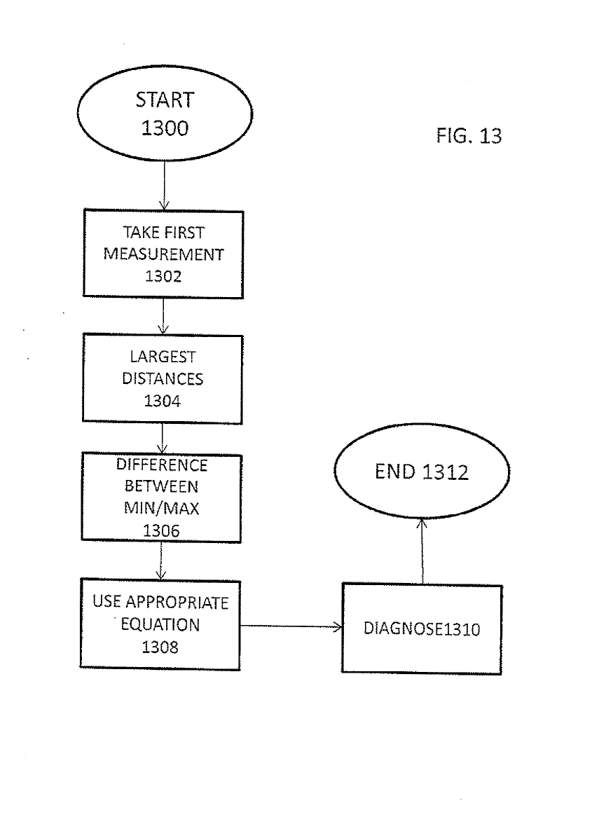

With particular reference to FIG. 13, there is illustrated a flow chart illustrating the relation of a thermography device to the body chemistry vulnerability index. The BCV, as noted above consists of a single number from one to ten and is based off the initial measurement 20. First, the initial measurement 20 is taken (1302). Once that data is collected, observe the data relating to the multi-site regions 23 for the head, the neck, the chest, the upper abdomen, the lower abdomen, and the back. In each of these regions 23, find the greatest delta in temperature between sites, which should result in six numbers (1304). Once the six greatest deltas are obtained, find the greatest delta amongst the deltas (Ex: if the six deltas were 1.5, 3.2, 2.1, 5.4, 7.8, and 4.9, the greatest delta amongst those would be 7.8-1.5 or 6.3) (1306). Next, insert the greatest delta amongst deltas into the appropriate equation provided in Table 2, which provides the result (1308). Once a technician has the index figure, this figure provides a one to ten scale of severity for the risk for the body to develop harmful conditions. A higher score on the BCV indicates that it is very likely that the current body chemistry of the patient is conducive to developing harmful conditions.

What is claimed is:

1. The method of diagnosing medical conditions comprising:
taking the body temperature of a plurality of body sites on a patient a first time thereby creating a first set of data;
subjecting the patient's body to a stimulus;
taking the body temperature of a plurality of the same body sites on a patient a second time thereby creating a second set of data;
comparing the first and second sets of data;
diagnosing a selected condition in group A, which heretofore was previously undiagnosable with a thermography device.

2. The method of claim 1, wherein the selected condition is a toxicity index.

3. The method of claim 1, wherein the selected condition is a deficit in detoxification capability.

4. The method of claim 1, wherein the selected condition is body chemistry vulnerability with an immune system deficiency.

5. The method of claim 1, wherein the selected condition is adrenal stress.

6. The method of claim 1, wherein the selected condition is an autoimmune disease.

7. The method of claim 1, wherein the selected condition is brain toxicity.

8. The method of claim 1, wherein the selected condition is thyroid hyperfunction or anomaly.

9. The method of claim 1, wherein the selected condition is hepatobiliary system stress.

10. The method of claim 1, wherein the selected condition is enzyme dysfunction or deficiency.

11. The method of claim 1, wherein the selected condition is insulin resistance.

12. The method of claim 1, wherein the selected condition is a pancreas dysfunction.

13. The method of claim 1, wherein the selected condition is a pelvic disorder.

14. The method of claim 1, wherein the selected condition is endometriosis.

15. The method of claim 1, wherein the selected condition is kidney hypo-function.

16. The method of claim 1, wherein the selected condition is a mastopathy.

17. The method of claim 1, wherein the severity of the selected condition is graded.

18. The method comprising:
taking the body temperature of a plurality of body sites on a patient a thereby creating a data set;
diagnosing susceptibility to harmful medical conditions in humans in the patient.

19. The method comprising:
taking the body temperature of a plurality of body sites on a patient a first time thereby creating a first set of data;
subjecting the patient's body to a stimulus;
taking the body temperature of a plurality of the same body sites on a patient a second time thereby creating a second set of data;
comparing the first and second sets of data;
assessing the preparedness of body systems or regions to respond to stimuli.
